(19) United States
(12) Patent Application Publication
(10) Pub. No.: US 2009/0291508 A1
(43) Pub. Date: Nov. 26, 2009

(54) NANOPARTICLES IN DIAGNOSTIC TESTS

(75) Inventors: Uma Mahesh Babu, Bradenton, FL (US); Robert W. VanDine, Montoursville, PA (US); Robert P. Sambursky, Bradenton, FL (US)

Correspondence Address: BROWN & MICHAELS, PC;400 M & T BANK BUILDING, 118 NORTH TIOGA ST, ITHACA, NY, 14850, US

(73) Assignee: Rapid Pathogen Screening Inc., Sarasota, FL (US)

(21) Appl. No.: 12/469,207

(22) Filed: May 20, 2009

Related U.S. Application Data
(60) Provisional application No. 61/071,833, filed on May 20, 2008.
(60) Provisional application No. 61/060,258, filed on Jun. 10, 2008.
(60) Provisional application No. 61/098,935, filed on Sep. 22, 2008.
(60) Provisional application No. 61/179,059, filed on May 18, 2009.

(51) Int. Cl. G01N 33/543; B01J 19/00

(52) U.S. Cl. 436/518; 422/61; 422/68.1; 977/904

(57) ABSTRACT

The present invention includes methods and devices that detect target molecules in a biological sample. The sample analysis device of the present invention includes nanoparticles. In one embodiment, the nanoparticles are directly immobilized on the surface of the sample analysis device. In another embodiment, the nanoparticles are indirectly immobilized on the surface of the sample analysis device by incorporating them in appropriate media and immobilizing the nanoparticles within a matrix.

### REFERENCE TO RELATED APPLICATIONS

This application claims one or more inventions which were disclosed in Provisional Application No. 61/071,833, filed May 20, 2008, entitled “NANOPARTICLES IN DIAGNOSTIC TESTS”. Provisional Application No. 61/060,258, filed Jun. 10, 2008, entitled “COMBINED VISUAL/FLUORESCENCE ANALYTE DETECTION TEST”, Provisional Application No. 61/098,935, filed Sep. 22, 2008, entitled “1N SITU LYSIS OF CELLS IN LATERAL FLOW IMMUNOASSAYS, and Provisional Application No. 61/179,059, filed May 18, 2009, entitled “METHOD AND DEVICE FOR COMBINED DETECTION OF VIRAL AND BACTERIAL INFECTIONS”. The benefit under 35 USC §119(e) of the United States provisional applications is hereby claimed, and the aforementioned applications are hereby incorporated herein by reference.

### BACKGROUND OF THE INVENTION

1. Field of the Invention

The invention pertains to the field of immunoassays. More particularly, the invention pertains to immunoassays that include nanoparticles that bind target molecules collected from a biological sample.

2. Description of Related Art

A number of antibody-based immunoassays are utilized in diagnostics for various diseases. These immunoassays combine various reagents and process steps to provide a sensitive and rapid means for the detection of target molecules. Immunoassays are available for a wide area of target analytes. The first tests were made for human chorionic gonadotropin (hCG). Today, there are commercially available tests for monitoring ovulation, detecting infectious disease organisms, analyzing drugs of abuse and measuring other analytes important to human physiology. Products have also been introduced for veterinary testing, environmental testing and product monitoring.

U.S. Pat. No. 5,714,341, incorporated herein by reference, discloses a lateral flow immunoassay for HIV specific antibodies in saliva samples. The saliva sample is diluted in a sample buffer and the lateral flow immunoassay is dipped into the diluted saliva sample.

German Patent DE 196 22 503, incorporated herein by reference, suggests using lateral flow immunoassays for the detection of illegal narcotics in saliva or sweat.

Immunoassays take advantage of the specific binding of an antibody to its antigen, however the use of immunoassays is limited by the availability of antibodies for the specific target, degradation of the antibodies, strength of binding to the antigen, and the cost of producing antibodies. Further, the temperature range of detection is limited by the thermal stability of the antibody. The sample matrix is often limited to a few biological fluids that are suitable for maintaining both the stability of the antibody and the affinity of the antibody for the antigen. Although a number of antibodies have been developed to rapidly detect various diseases, a need exists for better alternatives.

U.S. Pat. No. 6,521,736, herein incorporated by reference, discloses polymeric micelle nanoparticles designed to bind to viruses at multiple sites. These polymeric micelles, referred to as “nanoviricides”, can be covalently linked to ligands that can specifically bind to the target viruses. Nanoviricides have been shown to bind specific viruses and “disassemble” the viral coating or deliver specific toxic drugs either to the viral surface or penetrate into the virus. The described therapeutic scenarios include disassembly of the viral coat and destruction of the virus by delivering drugs as described above, as well as engulfing or coating the surface of the viral target, neutralization of viral ability to bind to normal cell receptors and destabilization of the viral surface.

### SUMMARY OF THE INVENTION

The present invention includes methods and devices that detect target molecules in a biological sample. The sample analysis device of the present invention includes nanoparticles. In one embodiment, the nanoparticles are directly immobilized on the surface of the sample analysis device. In another embodiment, the nanoparticles are indirectly immobilized on the surface of the sample analysis device by incorporating them in appropriate media and immobilizing the nanoparticles within a matrix.

### BRIEF DESCRIPTION OF THE DRAWINGS

FIG. 1 shows a sample analysis device in an embodiment of the present invention.

FIG. 2 shows a housing containing the strip of FIG. 1.

FIG. 3 shows a collection device for collecting a sample.

FIG. 4 shows a test kit including the sample analysis device of FIGS. 1 and 2 and the collection device of FIG. 3.

FIG. 5 shows another embodiment of a sample analysis device of the present invention.

FIG. 6 shows a sample zone and a test line of a sample analysis device containing nanoparticles in an embodiment of the present invention.

FIG. 7A shows an example of the nanoparticles bound to a target in the sample zone according to the methods and devices of the present invention.

FIG. 7B shows the example of FIG. 7A, at the test line, as well as the readout of this example on a sample analysis device of the present invention.

### DETAILED DESCRIPTION OF THE INVENTION

The present invention provides a sensitive and rapid method for the detection of targets e.g. organisms, pathogens and/or molecules in a sample using nanoparticles. Samples potentially containing targets are applied onto a sample analysis device, on which an analysis of the targets, e.g. by chemical or biochemical means can take place. Further, a test kit and a composition for carrying out the method of the invention are provided.

The invention provides a sensitive and rapid method for the detection of targets in a mobile phase. The targets are selected from organisms, cells, e.g. normal and benign abnormal cells, viruses, fungi, bacteria, parasites, tumors, hormones, prions, allergy-associated components, toxins, and drugs. Preferably, the method comprises a parallel determination of a plurality of targets.

As conventionally defined, nanoparticles are any particles that are nanometers in size. In the present invention, however, polymeric micelles, optionally covalently or non-covalently attached to other moieties as described below, will hereinafter be referred to as “nanoparticles”. Polymeric micelles are nano-sized carriers that water-solubilize hydrophobic drug molecules. These polymers have a branched, hydrophobic interior (core) and hydrophilic exterior (shell) to maintain the physical properties characteristic of conventional micelles, but have enhanced thermodynamic stability. The shapes of the polymeric micelles may include, but are not limited to, spherical fullerenes (also known as bucky balls) and other geodesic domes, as known in the art.

The nanoparticles used in the present invention are rationally designed to bind to target molecules collected from a biological sample. The nanoparticles are immobilized (directly or indirectly) on the surface of a sample analysis device. The surface may be a protein binding membrane of materials such as nitrocellulose, nylon, polyester, or polystyrene. The sample collection material may include, for instance, Dacron® fibers, polyester, glass fiber, cellular acetate or a nylon mesh.

Preferred targets include, but are not limited to, proteins, glycoproteins, proteoglycans, lipoproteins and lectins. In preferred embodiments, the target is on the surface of the cell or organism, so it is not necessary to lyse the cells to detect the target.

In some preferred embodiments, the sample that has been collected is not lysed prior to collection and transfer to the sample analysis device. This decreases the number of steps needed to collect and prepare the sample for analysis. Following sample loading, the sample traveling with the transport liquid (buffer) will encounter a lysis agent. The lysis agent will have preferably been pre-loaded and dried onto the test strip and is eluted by the transport liquid. The initially dried lysis agent is preferably localized between the sample application zone and a conjugate zone. The lysing agent is preferably soluble in the sample transport liquid, and the lysing agent is solubilized and activated upon contact with the sample transport liquid. The sample transport liquid then contains both lysing agent in solution or suspension and sample components in suspension. Any lysis-susceptible components in a sample, then being exposed in suspension to the lysing agent, are themselves lysed in situ. The running buffer then carries the analyte, including any lysis-freed components, to a detection zone.

The location where the lysis agent is pre-loaded and dried can be varied as needed. In order to maximize the time that the sample has to interact with the lysis agent as well as to minimize the amount of lysis agent reaching the detection zone, the dried lysis agent may be located in or just downstream of the sample application zone. Or, in order to minimize the distance along which the lysis product must travel before reaching the conjugate zone, the dried lysis agent may be located closer to the conjugate zone.

Lateral flow devices are known, and are described in, e.g., U.S. Published Patent Application Nos. 2005/0175992 and 2007/0059682, the contents of which are incorporated by reference. Other lateral flow devices known in the art could alternatively be used with the systems and methods of the present invention.

In one embodiment, the nanoparticles are directly immobilized on the surface of a sample analysis device. For direct immobilization, the nanoparticles may include chemical moieties such as carboxyl, amino, hydroxyl or sulfhydryl groups that can bind directly onto the protein binding membrane, which is nitrocellulose in some embodiments. The nanoparticles can be conjugated to peptides, proteins or other linkages that can directly bind to the nitrocellulose. Examples of such “linkers” include, but are not limited to, poly-lysine, peptides, proteins etc.

In another embodiment, the nanoparticles are indirectly immobilized on the surface of the sample analysis device by incorporating them in media to immobilize the nanoparticles within a matrix. Some media that could be used include, but are not limited to, hydrogels, polystyrene beads, and other particles or media that immobilize the nanoparticles within a matrix. The nanoparticles are thus indirectly bound on the surface of the sample analysis device, but still have “access” to analytes in the lateral fluid flow. Preferably, the nanoparticles are mixed with gels, including but not limited to, hydrogels and colloidal gels prior to, during, or after application to the sample analysis device.

In one embodiment, the nanoparticles used in the diagnostic tests of the present invention are those disclosed in U.S. Pat. No. 6,521,736, herein incorporated by reference, and include a structure represented by:

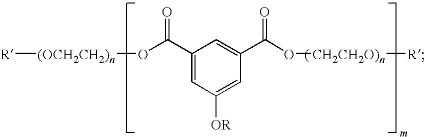

wherein

R is hydrogen, a linear or branched alkyl group, a linear or branched alkenyl group, or an aryl group;

wherein said alkyl, alkenyl, or aryl group is unsubstituted or substituted with one or more heteroatomic functional groups; R′ is hydrogen, an acyl group, an antibody fragment, a chemomimetic functional group, an immunoconjugate, a ligand for a biological target, or

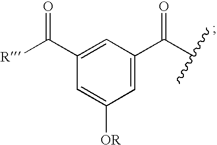

wherein

R′″ is a hydroxyl group, an alkoxyl group, or a primary or secondary amino group;

n is at least 1; and m is at least 1.

The nanoparticles preferably have terminal or internal hydroxyl, amino, carboxyl, or sulfhydryl groups that can provide attachment points for epitopes, antibody fragments, chemomimetic functional groups, ligands for biological targets, and small ligands known to bind to receptors on specific targets including, but not limited to, organisms, cells, including normal and benign abnormal cells, tissues, viruses, fungi, bacteria, tumors, hormones, carbohydrates, enzymes, receptors, DNA fragments, prions, and parasites. Furthermore, the nanoparticles can also be conjugated with tags including, but not limited to, visible or colored particles, dyes, magnetic particles, fluorescent particles, phosphorescent particles, chemiluminescent particles, radioisotopic ligands, enzymes, peptides, amino acids, colloidal particles, or beads.

The nanoparticles are preferably designed according to the methods described in U.S. Pat. No. 6,521,736 to specifically bind to labels, dyes, sample analytes, albumin, or linking agents that bind the nanoparticles at the test or control lines.

In other embodiments, the nanoparticles that may be used include, but are not limited to, selenium, carbon, and colloidal gold.

The nanoparticles can range from 1 to 100 nanometers in size, preferably 3 to 75 nanometers, and more preferably 5 to 50 nanometers.

The sample to be analyzed can be obtained from a variety of biological or environmental sources including, but not limited to, animal, human, plant, fungal, ecological, agricultural, and industrial sources. Examples of some applications of the diagnostic tests of the invention include detection of pathogens, infections including fungal, viral and bacterial, allergens, hormones, cancers, parasites, industrial pollutants, toxins, or toxic waste.

The invention may be performed by means of a simple test kit. By eliminating the need for the classical “antigen” and “antibody” binding, the present invention overcomes the limitations of immunoassay tests currently used in the art of diagnostic testing.

Another important advantage of the invention is that nanoparticles are highly stable in both solution and solid phases, with a shelf-life that is much longer (5 to 20 years) than currently available diagnostic tests because there is no need to use antibodies which tend to degrade over short periods of time, typically less than 1 to 3 years. Therefore, the disclosed method is more stable and longer lasting than the immunoassay methods of the prior art. Furthermore, due to the thermal stability of the nanoparticles, such tests can be conducted at wider temperature ranges than the methods of the prior art. The test kits of the present invention can also be produced, transported and stored in a wider variety of temperatures and conditions than antibody-based test kits.

The nanoparticles are designed to bind to specific surface markers on the targets. Thus, a further advantage of the present invention includes elimination of sample preparation steps since bacteria and viruses in a sample do not need to be lysed prior to detection. Furthermore, since antibodies are not used in the assay, the cost of producing antibodies, as well as the problem of antibody degradation, is avoided. In addition, while only a few biological fluids are practicable matrices for antibody-based immunoassays, the present invention can detect targets in any matrix where the target is found. Additional cost savings are realized by manufacturing single or multiple formulations of the specially designed nanoparticles like those in a panel such as Hepatitis panel (Hepatitis A, B, C etc,), cardiac panel, sexually transmissible infection panel, neurological panel, respiratory panel etc. where different members of the panel or closely related members are simultaneously detected and/or differentiated.

In a preferred embodiment, the present invention provides for the reduction of interfering substances that might be present in the sample to be tested. Since an interfering substance, e.g. a human anti-mouse antibody (HAMA), may also be capable of forming a complex with the labeled, non-immobilized reagent of the reagent zone and the immobilized binding partner of the detection zone, thus indicating a positive test result in the immunoassay, the carrier may further comprise at least one capturing zone. Each capturing zone contains an immobilized capturing reagent specifically binding to a certain interfering substance, thereby immobilizing the interfering substance in the capturing zone. As the capturing zone is separated from the detection zone by space, and the sample starts to migrate over the reagent zone and the capturing zone before reaching the carrier's detection zone, the method allows a separation of the interfering substance or substances from the analyte or analytes of interest. Preferably, the capturing zone is located between the reagent zone and the detection zone. However, the capturing zone may also be located between the application zone and the reagent zone.

The invention also discloses a point-of-care method for detection of targets. The method is suitable for diagnosis in human beings, plants and animals, e.g. pets or livestock animals. A preferred application is the detection of pathogens in a biological fluid. For example, the pathogen is selected from the group of viruses, fungi, and bacteria and combinations thereof.

Examples of viral pathogens include, but are not limited to, retroviruses, adenoviruses, herpesviruses, cytomegaloviruses, hepatitis viruses, dengue, influenza viruses, parainfluenza, papilloma viruses, rotavirus, human immunodeficiency virus (HIV), feline immunodeficiency virus (FIV) coxsackie, enterovirus, marburg, ebolavirus, Epstein Barr Virus, respiratory syncytial virus, echovirus, meningitis, lyssavirus, foot and mouth disease virus, rabies, pseudorabies, viral pneumonia, West Nile virus, parvoviruses, feline leukemia virus (FeLV), Rous sarcoma virus (RSV), Norwalk virus, rhinoviruses, Rubella, Astrovirus, Varicella-Zoster, and Metapneumovirus.

Examples of bacterial pathogens which can be detected by the invention include, but are not limited to, Chlamydia species, Mycoplasma species, Proteus, Anthrax, Clostridium species, Salmonella species, Pseudomonas species, Shigella species, Hemophillus species, Campylobacter species, Mycobacterium and Atypical Mycobacterium species including leprosy, avium, chelonae and tuberculosis, Streptococcal species, Staphylococcal species, Neisseria species, helicobacter, Escherichia coli species, Brucella species, Rickettsial species, Gardenella, Borrelia species, Diphtheria species, trichomonas, Toxoplasma species, Moraxella species, Bordetella species, Treponema species, and Legionella species.

In addition, the invention provides a method for detection of cancers and tumors including, but not limited to, breast, prostate, skin, nasal pharyngeal, lung, brain, pancreatic, leukemic, colorectal, ovarian, cervical, lymphoma, or intraocular cancers or tumors. The invention further provides a method for detection of allergens, chagas, leishmania, hormones, normal cells, benign abnormal cells, fungi, dust mites, storage mites, cytokines, chemokines, acute phase reactants, complement, erythrocytes, leukocytes, macrophages, dendritic cells, stem cells, plant diseases, malaria, leptospira, giardia, syphilis, Orions, cryptosporidia, heartworms, and plant cells.

The invention has applications for diagnostic testing in the medical, veterinary, agricultural, industrial, environmental, forensic, biothreat, agrothreat, and chemothreat fields.

In a preferred embodiment, the sample analysis device includes a chromatographic test strip, e.g. a lateral flow or flow through test strip. The test strip includes a sample application zone and a detection zone. Preferably, the test strip also includes a waste zone, a control zone, a carrier backing, a housing and an opening in the housing for result read out. Any combinations of some or all of these elements may be included in the test strip. Sample analysis in the detection zone may be carried out by standard means, e.g. by a biochemical or enzymatic detection method. Preferably, the detection method includes the use of nanoparticles capable of specifically binding the targets, e.g. pathogens to be tested and subsequent visualization of the bound entity, e.g. by enzymatic detection or by means of direct labeling groups, such as visible or colored particles, dyes, magnetic particles, fluorescent or phosphorescent particles, chemiluminescent particles, radioisotopic ligands, enzymes, peptides, amino acids, colloidal particles, or beads, as is well known in the art.

Detection of the marker may be achieved in the detection zone. The binding molecule immobilizes the labeled complex or the labeled marker-analogue by immune reaction or other reaction in the detection zone, thus building up a visible test line in the detection zone during the process. Preferably, the label is an optically detectable label. Forming a complex at the test line concentrates and immobilizes the label and the test line becomes visible to the naked eye, indicating a positive test result. Particularly preferred are direct labels, and more particularly gold labels which can be best recognized by the naked eye. Additionally, an electronical read out device (e.g. on the basis of a photometrical, acoustic, impedimetrical, potentiometric and/or amperometric transducer) can be used to obtain more precise results and a semi-quantification of the analyte. Other labels may be latex, fluorophores or phosphorophores.

In one embodiment, the sensitivity of visually read lateral flow immunoassay tests is enhanced by adding a small quantity of fluorescing dye or fluorescing latex bead conjugates to the initial conjugate material. When the visible spectrum test line is visibly present, the test result is observed and recorded. However, in the case of weak positives that do not give rise to a distinct visual test line, a light of an appropriate spectrum, such as a UV spectrum, is cast on the test line to excite and fluorescent the fluorescing latex beads which are bound in the test line to enhance the visible color at the test line.

In another embodiment, the nanoparticles can be used in formulations such as spraying solutions and hence can be sprayed onto targets, e.g. on a surface or in the air to detect the target of interest. The nanoparticles in the spray bind to the organism present on the surface and form the complex that can be detected as described above.

Furthermore, this invention includes a device and test kit for the performance of the described method.

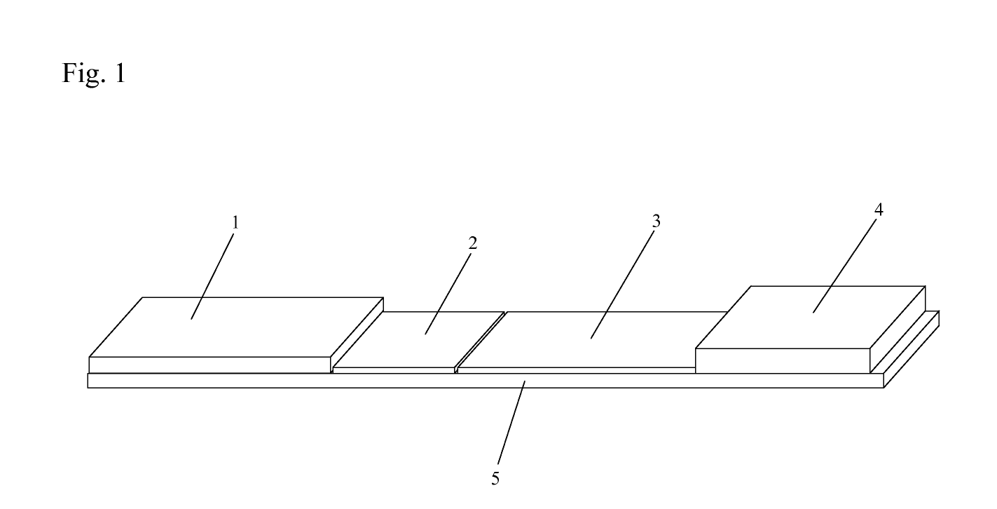

US Patent Publication No. 2005/0175992, published Aug. 11, 2005, discloses examples of a sample analysis device, which could be used in the present invention. This application is incorporated herein by reference. The chromatographic test strip shown in FIGS. 1 through 4 includes a plurality of different strip materials. The device preferably includes an absorbent pad (1), an application zone (2), a detection zone (3) and a waste zone (4). The strip materials are arranged on an adhesive plastic backing (5). The absorbent pad (1) is provided in this example for adding an elution medium in order to facilitate the transfer of the sample to the detection zone (3). US Patent Publication No. 2007/0059682, published Mar. 15, 2007, also incorporated herein by reference, describes methods to increase specificity of lateral flow immunoassays. These methods could also be used in combination with the embodiments described herein.

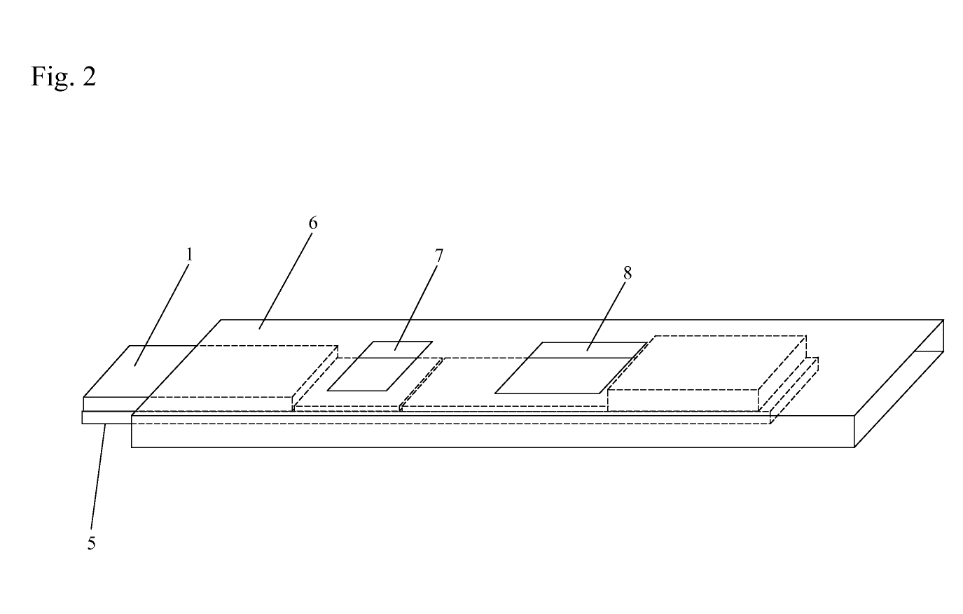

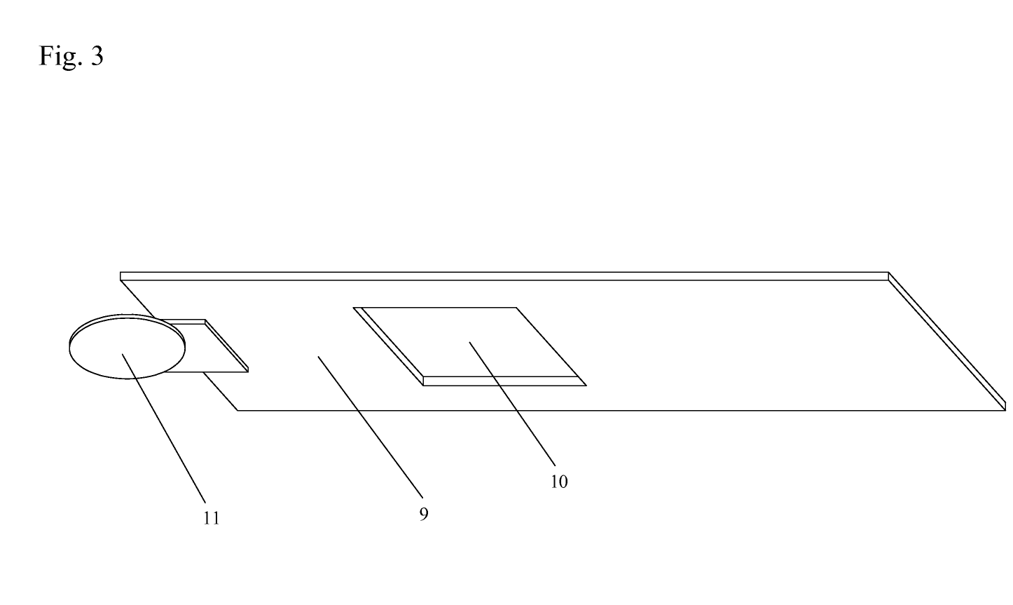

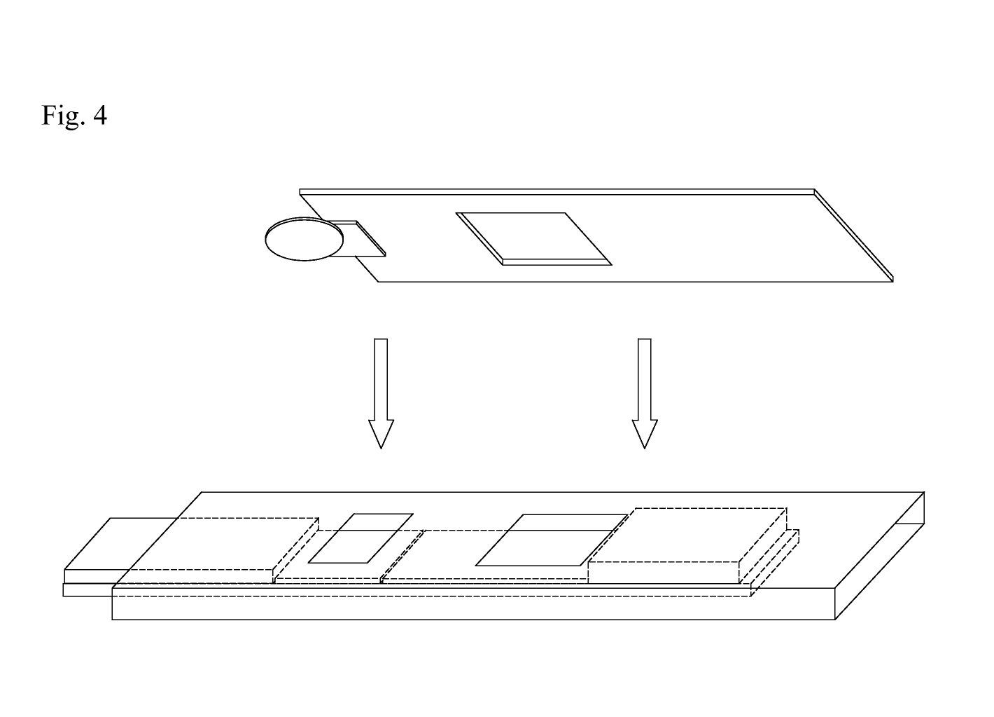

FIG. 2 shows a housing (6), which is preferably plastic, containing the strip as shown in FIG. 1. A sample application window (7) brings a collection device into contact with the strip. The test result is displayed in the read out window (8). FIG. 3 shows the collection device for collecting a sample. In one example, the collection device is a swab member. The collection device includes a body (9), which is preferably plastic, with a sample collection material (11) fixed on it and an opening (10) corresponding to a read out window when the collection device is operatively in contact with a test strip. FIG. 4 shows a test kit, which includes the sample analysis device of FIGS. 1 and 2 and the collection device of FIG. 3.

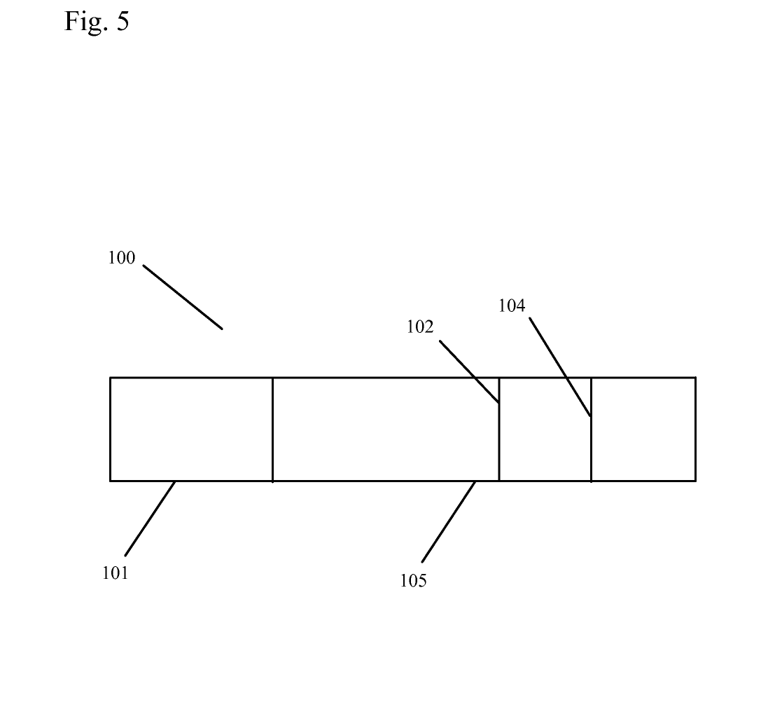

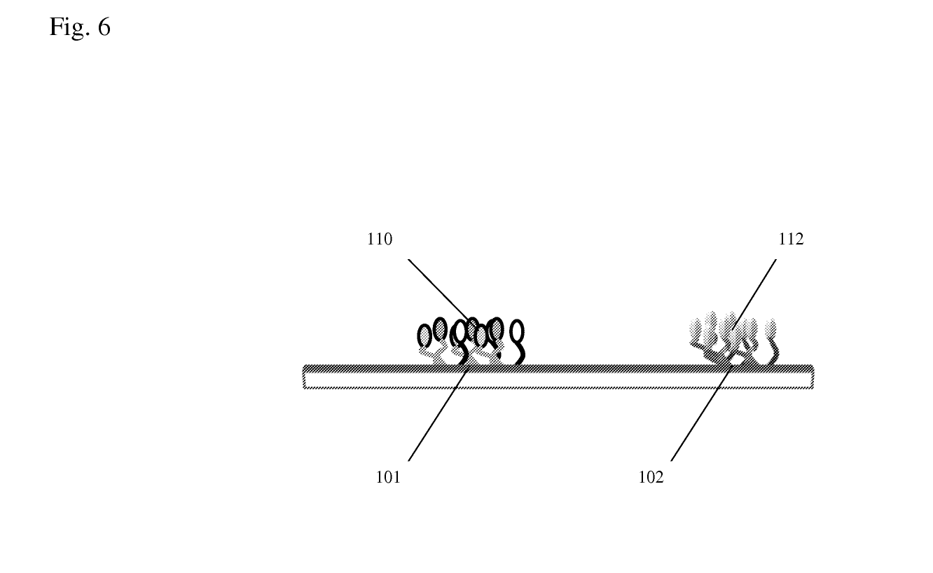

In a method of the invention, it is possible to make use of different biochemical testing procedures to detect constituents on one or several biochemical binding reactions. In a preferred embodiment, as shown in FIG. 5 and FIG. 6, the chromatography test strip (100) includes an application zone (or sample zone) (101). The sample is applied to the application zone (101). In some embodiments, as shown in FIG. 6, the application zone (101) includes nanoparticles (110) with a detectable tag. For example, the nanoparticles (110) may be dyed with a visible color to create color at the test line (102) for a positive test.

The test strip also includes a detection zone (105). The detection zone (105) includes a test line (102) that preferably contains at least one nanoparticle (112) that is immobilized (either directly or indirectly) on the protein binding surface. Although only one test line is shown in the figure, multiple test lines are within the spirit of the invention. In some embodiments where there are multiple targets, the presence of each target preferably corresponds to a separate test line. In other embodiments where there are multiple targets, the presence of multiple targets may be indicated on the same test line such that the presence of more than one target has different characteristics than the presence of a single target. For example, the presence of multiple targets on the same test line may be visually indicated by a different color than the presence of each of the targets alone.

The nanoparticles are capable of specifically binding to an analyte and to a further specific reagent in the detection zone (105). The detection zone (105) also preferably includes a control section, which includes a control line (104) indicating the functionality of the test. Although only one control line is shown in the figure, multiple control lines may alternatively be used.

In a preferred embodiment, the control line (104) includes a recombinant protein which binds to a component of the elution medium or other composition being used in the test. In one example, the recombinant protein is a lectin. Lectins are specific binding agents for different proteins. Lectins are complex molecules containing both protein and sugars. Lectins bind to the outside of a cell and can cause biochemical changes in the cell. This family of animal proteins binds very specifically to particular sugar residues of glycoproteins. Different lectins are specific for certain materials, for example, some bind to albumins, while others may bind to red blood cell or white blood cell membranes. Particular lectins can be chosen for use in the present invention based on their affinities for glycoproteins that may be found in the running buffer or other composition being used in the methods and devices of the present invention.

In one preferred embodiment, Albegone recombinant protein, a lectin which binds to all mammalian albumins, may be used. Albegone recombinant protein is a proprietary recombinant protein from Advanced Product Development and Consulting for the Life Sciences, State College, Pa.

Albumins, such as Bovine Serum Albumin (BSA) and the human counterpart HSA are often used in point of care devices in a variety of ways. For example, the albumins may be in the running buffer, to block the unreacted sites on nitrocellulose, in the conjugate or sample zones or in the conjugate itself. In a preferred embodiment, nanoparticles bound to albumin (control conjugate) are mixed with the test conjugate (sample conjugate) of nanoparticles. This mixture of test conjugate and control conjugate give rise to the test and control lines.

The mobile phase travels on the test strip due to at least one force including, but not limited to capillary action, vacuum action, and gravity. Preferably, fluid is transported by capillary action.

In a preferred embodiment, the nanoparticles are conjugated to specific binding partners for the analytes in the detection zone. The specific binding partner can be selected from epitopes, antibody fragments, chemomimetic functional groups, ligands for biological targets, and small ligands capable of binding to a target. Other types of binding partners are bioorganic molecules like aptamers or receptors.

While the detection zone is shown after the sample application zone in the figures, the detection zone is preferably located within or after the sample application zone, seen in the running direction of the eluent liquid. The test line is located after the application zone and the control line is located after the test line. Together, the test line and control line make up the detection zone.

The control zone preferably includes a recombinant protein such as a lectin immobilized on the sample analysis device at the control line. In an example where a lectin with an affinity for albumins is used, albumin is added to the sample and the mobile phase includes the sample, nanoparticles which are rationally designed to bind to albumin and are labeled with a detectable tag, as well as labeled nanoparticles which are rationally designed to bind to the analyte. Once the mobile phase travels downstream on the test strip and reaches the control zone, the albumin binds to the lectin and the control line appears, indicating the correct flow characteristics of the immune-chromatography test.

Depending on the type of detection method, different binding partners are present in the different zones. In a sandwich immunoassay, it is preferred to have an immobilized analyte binding partner in a conjugate zone. The binding partner forms a complex with the analyte and thereby immobilizes the binding partner at the test line. In a preferred manner, nanoparticles are immobilized on a sample analysis device forming the bottom layer of the sandwich. More preferably the bottom layer nanoparticle conjugates are in colloidal form and have an intrinsic “black” color. The analyte includes the middle layer of the sandwich where the sample to be detected is bound by nanoparticles including the top and bottom layers of the sandwich.

A mobile phase including nanoparticle conjugates, a control substance (such as albumin or another substance for which the recombinant protein being used on the control line has a specific affinity), and a suitable carrier forms the top layer of the “sandwich”. The nanoparticles in the mobile phase bind to analytes. Analytes also bind to the bottom layer nanoparticles and are thereby immobilized at the test line. Preferably, the mobile phase nanoparticle conjugates are labeled. More preferably, the label of the mobile phase nanoparticle conjugates is an optically detectable label. The control reaction product can be applied at any point. A waste zone downstream of the control line which is downstream of the test line is preferable to collect the excess mobile phase.

The carrier may be a buffer suitable for diagnostic purposes. Preferably the carrier is water, although organic carriers may be used if desired. In any event, the carrier should be a liquid which is inert to the reactants in the system.

In a preferred embodiment, the mobile phase, including labeled nanoparticle conjugates, the sample analyte, the control substance, and a suitable carrier, are first mixed and then applied to the application zone.

The top layer nanoparticle conjugates form a complex at the test line so that the test line is detectable by methods including, but not limited to, visual inspection, fluorescence detection, phosphorescence detection, chemiluminescence assay, enzymatic assay, radio assay, magnetic assay, agglutination, and ouchterlony. Particularly preferred are direct labels, and more particularly dyes which can be best recognized by the naked or unaided bare eye. Additionally, a read out device can be used to obtain more precise results and a semi-quantification of the analyte. The conjugate zone also contains similar nanoparticles attached to the control substance mixed with the test nanoparticle conjugates.

### EXAMPLES

The invention is further described in the following examples, which do not limit the scope of the invention described in the claims.

### Example 1

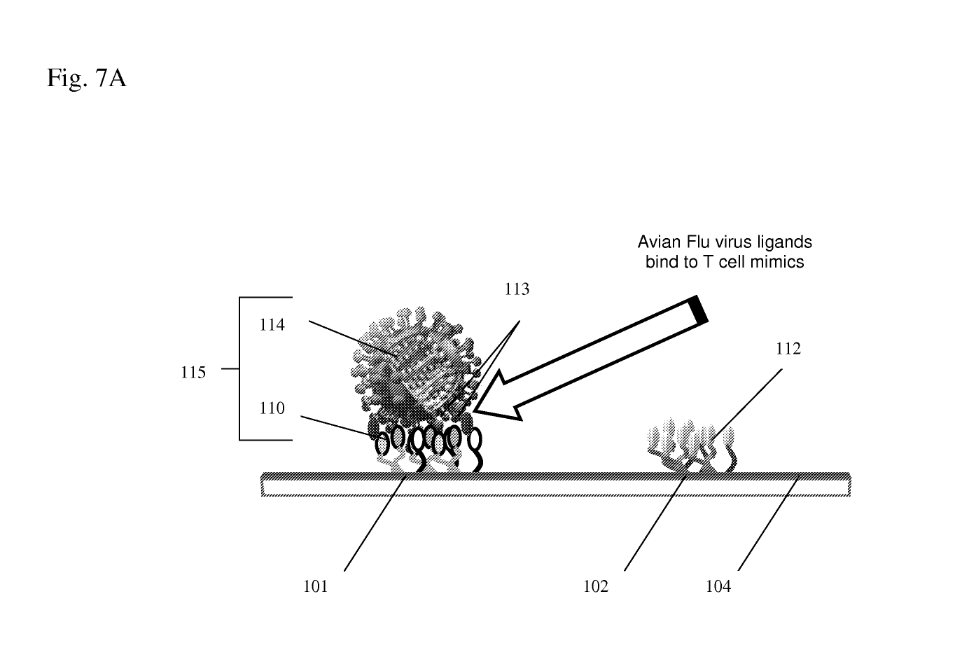

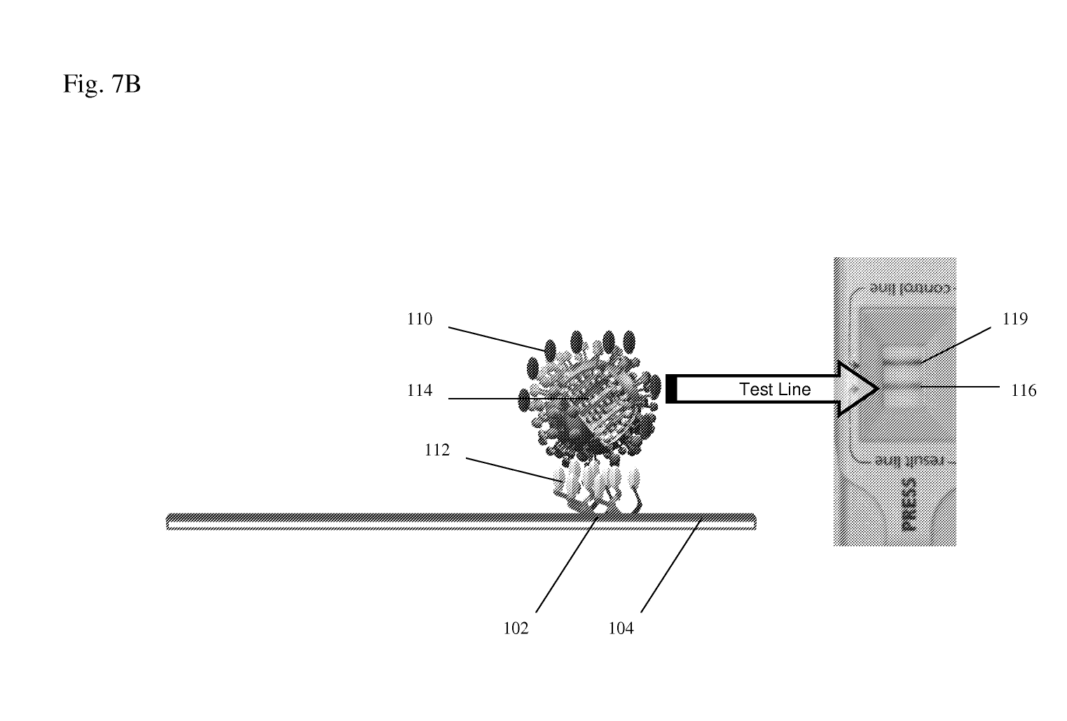

As shown in FIGS. 7A and 7B, and referring back to FIG. 6, in one example, the sample zone (101) of a sample analysis device is composed of nanoparticles (110) that mimic the action of T cells in the immune system (T cell Mimics). The “T cell mimics” preferably only include the active site (the binding site) for the viral particle of interest (H5N1 in this example). Since there are multiple binding sites, the device has a higher binding affinity and higher sensitivity for the target (a virus in this example) than in prior art devices. The “T cell mimics” are dyed with a visible color to create color at the test line for a positive test. The sample zone “T cell Mimics” may be specific to a single serotype or general to bind to all viruses.

At the test line (102), additional nanoparticles (112), which are also “T cell mimics”, are immobilized on the sample analysis device. These nanoparticles (112) are preferably colorless. The test line “T cell mimics” may be specific to bind to one serotype or general to bind all viruses.

The visual tag is attached to nanoparticles in the mobile phase in this example, and the colorless nanoparticles are immobilized on the test line. However, the visual tag could alternatively be attached to the nanoparticles immobilized on the test line, with colorless nanoparticles in the mobile phase.

The target viral particle in this example is Avian Flu (H5N1). Avian Flu is applied to the sample zone (101). The “T cell mimics” (110) in the sample zone (101) bind to the surface ligands (113) of the viral particle (114). The viral particle (114) and the “dyed T cell mimic” (110) form a bound complex (115) that is now tagged with a visual color. In this illustration the visual tags are red.

A running buffer moves the complex (115), which includes the visually tagged H5N1 Avian Flu viral particle (114), to the test line (102). The colorless “T cell mimics” (112) immobilized at the test line (102) bind to the available surface ligands (117) of the H5N1 Avian Flu viral particle (114). The accumulation of visually tagged H5N1 Avian Flu viral particles (114) form a visual read line (116) at the test line (102) location. There is also a visual read line (119) at the control line (104) location, indicating that the test is functioning.

### Example 2

A test kit includes a test strip for the detection of Adeno virus in a tear sample from a patient. The test strip in this example includes a nitrocellulose protein binding membrane. The application zone includes an accessible portion of the test strip upstream of a test line. The test line is upstream of a control line.

The detection zone includes a nitrocellulose (NC) membrane with a nominal pore size of 8 pm and a thickness of 100 pm produced by Schleicher & Schuell, Germany. The test line contains an immobilized nanoparticle and a ligand rationally designed to bind to Hexon protein or any specific viral surface marker and is hence specific for the surface marker epitope on the Adeno virus membrane. As long as the test is working, the control line will appear regardless of whether the sample is positive or negative and thus indicates the correct flow characteristics of the immune-chromatography test. The chromatographic zones are in fluid communication with each other in order to create a fluid pathway.

A tear sample from a patient is mixed with a mobile phase containing nanoparticles labeled with a visual color dye and/or a fluorescent dye tag and a ligand rationally designed to bind to Hexon protein that is specific for the surface marker epitopes on the Adeno virus membrane. The adenovirus is bound at the test line “sandwiched” between the colloidal bottom-layer nanoparticles and the labeled top-layer nanoparticles. The signal from adenovirus bound to labeled nanoparticles is then viewed with a naked eye or an “aided” eye under fluorescent lighting. The signal from albumin conjugated to labeled nanoparticles is observed at the control line.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method for the detection of at least one target, comprising the steps of:
a) transferring a sample to a sample analysis device, wherein the sample analysis device comprises a plurality of nanoparticles that bind to the target; and
b) analyzing the sample.

2. The method of claim 1, wherein the nanoparticles are conjugated to a specific binding partner for the target.

3. The method of claim 1, further comprising, prior to step a), the step of collecting the sample.

4. The method of claim 1, wherein the nanoparticles are directly immobilized on a surface of the sample analysis device.

5. The method of claim 1, wherein the nanoparticles are indirectly immobilized on a surface of the sample analysis device by incorporating them in media and immobilizing the nanoparticles within a matrix.

6. The method of claim 1, wherein the nanoparticles comprise a structure represented by: 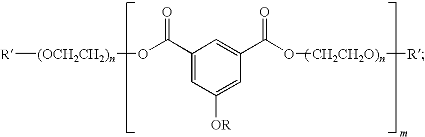
wherein
R is hydrogen, a linear or branched alkyl group, a linear or branched alkenyl group, or an aryl group;
wherein said alkyl, alkenyl, or aryl group is unsubstituted or substituted with one or more heteroatomic functional groups; R′ is hydrogen, an acyl group, an antibody fragment, a chemomimetic functional group, an immunoconjugate, a ligand for a biological target; or 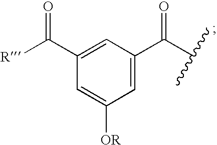
wherein
R′″ is a hydroxyl group, an alkoxyl group, or a primary or secondary amino group;
n is at least 1; and m is at least 1.

7. The method of claim 1, further comprising the step of mixing the sample with labeled nanoparticles prior to step a).

8. The method of claim 1, further comprising, prior to step a), the step of applying a spraying solution comprising a plurality of nanoparticles that bind to the target onto a surface or in the air where the target may be located.

9. The method of claim 8, wherein the nanoparticles in the spraying solution are labeled nanoparticles and the nanoparticles on the sample analysis device are nonlabeled nanoparticles.

10. The method of claim 1, wherein the sample analysis device comprises a sample application zone where the collected sample is applied to the sample analysis device, comprising labeled nanoparticles and a detection zone comprising unlabeled nanoparticles.

11. A test kit comprising:
a) a collection device for collecting a body-fluid sample; and
b) a sample analysis device comprising reagents for determining the presence and/or amount of at least one target, wherein the reagents comprise a plurality of nanoparticles that bind to the target.

12. The test kit of claim 11, wherein the nanoparticles are conjugated to a specific binding partner for the target.

13. The test kit of claim 11, wherein the nanoparticles comprise a structure represented by: 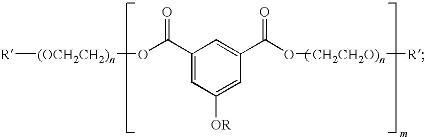
wherein
R is hydrogen, a linear or branched alkyl group, a linear or branched alkenyl group, or an aryl group;
wherein said alkyl, alkenyl, or aryl group is unsubstituted or substituted with one or more heteroatomic functional groups; R′ is hydrogen, an acyl group, an antibody fragment, a chemomimetic functional group, an immunoconjugate, a ligand for a biological target; or 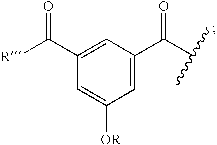
wherein
R′″ is a hydroxyl group, an alkoxyl group, or a primary or secondary amino group;
n is at least 1; and m is at least 1.

14. The test kit of claim 11, wherein the sample analysis device further comprises a sample application zone where the collected sample is applied to the sample analysis device.

15. The test kit of claim 14, wherein the sample application zone comprises labeled nanoparticles that bind to the target.

16. The test kit of claim 15, wherein the sample analysis device further comprises a detection zone, wherein the detection zone comprises unlabelled nanoparticles that bind to the target.

17. The test kit of claim 16, wherein the detection zone further comprises a control zone for determining if the test kit is properly functioning.

18. The test kit of claim 11, wherein the sample analysis device further comprises a detection zone, wherein the detection zone comprises nanoparticles that bind to the target.

19. The test kit of claim 18, wherein the detection zone further comprises a control zone for determining if the test kit is properly functioning.

20. The test kit of claim 11, wherein the nanoparticles are directly immobilized on a surface of the sample analysis device.

21. The test kit of claim 11, wherein the nanoparticles are indirectly immobilized on a surface of the sample analysis device by incorporating them in media and immobilizing the nanoparticles within a matrix.

22. A sample analysis device for detection of at least one target, comprising:
a) an application zone for applying a sample to the sample analysis device;
b) a detection zone for detecting the analyte; and
c) a plurality of nanoparticles that bind to the target.

23. The sample analysis device of claim 22, wherein the nanoparticles are located on the sample analysis device at a location selected from the group consisting of:
a) within the application zone;
b) within the detection zone;
c) within the application zone and within the detection zone;
d) between the application zone and the detection zone;
e) within the application zone and between the application zone and the detection zone; and
f) within the detection zone and between the application zone and the detection zone.

24. The sample analysis device of claim 22, wherein the nanoparticles comprise a plurality of labeled nanoparticles located within the application zone and a plurality of nonlabeled nanoparticles located within the detection zone.

25. The sample analysis device of claim 22, wherein the nanoparticles are conjugated to a specific binding partner for the target.

26. The sample analysis device of claim 22, wherein the nanoparticles are directly immobilized on a surface of the sample analysis device.

27. The sample analysis device of claim 22, wherein the nanoparticles are indirectly immobilized on a surface of the sample analysis device by incorporating them in media and immobilizing the nanoparticles within a matrix.

28. The sample analysis device of claim 22, wherein the nanoparticles comprise a structure represented by: 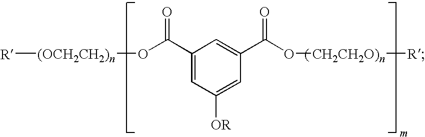
wherein
R is hydrogen, a linear or branched alkyl group, a linear or branched alkenyl group, or an aryl group;
wherein said alkyl, alkenyl, or aryl group is unsubstituted or substituted with one or more heteroatomic functional groups; R′ is hydrogen, an acyl group, an antibody fragment, a chemomimetic functional group, an immunoconjugate, a ligand for a biological target; or 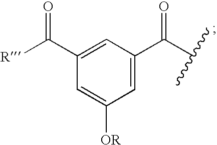
wherein
R′″ is a hydroxyl group, an alkoxyl group, or a primary or secondary amino group;
n is at least 1; and m is at least 1.
